(19) United States
(12) Patent Application Publication
(10) Pub. No.: US 2018/0235516 A1
(43) Pub. Date: Aug. 23, 2018

(54) Method and System for Determining Step Length

(71) Applicant: Veristride Inc., Salt Lake City, UT (US)

(72) Inventor: Stacy J. Morris Bamberg, Salt Lake City, UT (US)

(21) Appl. No.: 15/898,559

(22) Filed: Feb. 17, 2018

Related U.S. Application Data
(60) Provisional application No. 62/460,713, filed on Feb. 17, 2017.

(51) Int. Cl. A61B 5/11; A61B 5/00

(52) U.S. Cl. CPC A61B 5/112; A61B 5/1114; G01C 22/006; A61B 5/0015; A61B 5/6807

(57) ABSTRACT

Determining step lengths of an individual includes determining a relative time to be used in association with each of a plurality of heel strikes of an individual, and identifying the plurality of heel strikes. For each given identified heel strike of the plurality of identified heel strikes, a point in time that corresponds to the determined relative time is associated with the given identified heel strike. A foot speed associated with at least one of a left foot of the individual or a right foot of the individual is determined. The identified plurality of heel strikes, the associated point in time of each of the identified plurality of heel strikes, and the determined foot speed is then analyzed to determine a step length associated with at least one of the left foot of the individual or the right foot of the individual.

This application claims priority to and the benefit of U.S. Provisional Application No. 62/460,713, filed Feb. 17, 2017, and entitled METHOD AND SYSTEM FOR DETERMINING STEP LENGTH, the entire disclosure of which is incorporated herein by reference.

### BACKGROUND

Computer systems and related technology affect many aspects of society. Indeed, the computer system's ability to process information has transformed the way we live and work. Computer systems now commonly perform a host of tasks (e.g., word processing, scheduling, accounting, etc.) that prior to the advent of the computer system were performed manually. More recently, computer systems have been coupled to one another and to other electronic devices to form both wired and wireless computer networks over which the computer systems and other electronic devices can transfer electronic data. In addition, computing systems (including sensors) are now often being coupled to humans to allow for performing a variety of tasks. For instance, computer systems are now being used for identification of individuals (e.g., biometrics, including finger print scanners, retina scanners, facial recognition, and so forth), for health monitoring (e.g., heart rate sensors, pulse oximeters, and so forth).

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

At least some embodiments described herein relate to determining step lengths of an individual. For example, embodiments may include, determining a relative time to be used in association with each of a plurality of heel strikes of an individual. Embodiments may further include identifying the plurality of heel strikes, including at least a left heel strike and a right heel strike. Embodiments may also include, for each given identified heel strike of the plurality of identified heel strikes, associating a point in time to the given identified heel strike. The point in time may correspond to the determined relative time.

Embodiments may further include determining a foot speed associated with at least one of a left foot of the individual or a right foot of the individual. The foot speed corresponds to a stride associated with the left foot or a stride associated with the right foot. Embodiments may further include analyzing the identified plurality of heel strikes, the associated point in time of each of the identified plurality of heel strikes, and the determined foot speed. Embodiments may also include, based on the analysis of the identified plurality of heel strikes, the associated point in time of each of the identified plurality of heel strikes, and the determined foot speed, determining a step length associated with at least one of the left foot of the individual or the right foot of the individual.

In this way, one or more components for determining step length may be coupled to an individual via footwear (e.g., shoes, boots, socks, and so forth) of the individual. The one or more components and potentially, one or more components separate from the footwear, may gather and analyze data associated with a determined foot speed of the individual, identified heel strikes of the individual, and a determined relative time for determining when the identified heel strikes occurred in relation to other identified heel strikes. This data may then be analyzed to determine both a left step length of the individual, as well as a right step length of the individual. The determined step lengths may then be utilized to determine when asymmetric step lengths may be causing chronic overloading of a particular leg of the individual when compared to the other leg of the individual. Such determinations may then allow for taking preventative and/or corrective measures associated with chronic overloading and any possible resulting issues (e.g., osteoarthritis).

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an example computer architecture that facilitates operation of the principles described herein.

FIG. 2 illustrates an example environment for determining step lengths of an individual.

FIG. 3 illustrates an example of spatial differences associated with a series of identified heel strikes of an individual.

FIG. 4 illustrates an example of temporal differences associated with a series of identified heel strikes of an individual.

FIG. 5 illustrates an example of both spatial and temporal differences associated with a series of identified heel strikes of an individual.

FIG. 6 illustrates a flowchart of a method for determining step lengths of an individual.

### DETAILED DESCRIPTION

At least some embodiments described herein relate to determining step lengths of an individual. For example, embodiments may include determining a relative time to be used in association with each of a plurality of heel strikes of an individual. Embodiments may further include identifying the plurality of heel strikes, including at least a left heel strike and a right heel strike. Embodiments may also include, for each given identified heel strike of the plurality of identified heel strikes, associating a point in time to the given identified heel strike. The point in time may correspond to the determined relative time.

Embodiments may further include determining a foot speed associated with at least one of a left foot of the individual or a right foot of the individual. The foot speed corresponds to a stride associated with the left foot or a stride associated with the right foot. Embodiments may further include analyzing the identified plurality of heel strikes, the associated point in time of each of the identified plurality of heel strikes, and the determined foot speed. Embodiments may also include, based on the analysis of the identified plurality of heel strikes, the associated point in time of each of the identified plurality of heel strikes, and the determined foot speed, determining a step length associated with at least one of the left foot of the individual or the right foot of the individual.

In this way, one or more components for determining step length may be coupled to an individual via footwear (e.g., shoes, boots, socks, and so forth) of the individual. The one or more components and potentially, one or more components separate from the footwear, may gather and analyze data associated with a determined foot speed of the individual, identified heel strikes of the individual, and a determined relative time for determining when the identified heel strikes occurred in relation to other identified heel strikes. This data may then be analyzed to determine both a left step length of the individual, as well as a right step length of the individual. The determined step lengths may then be utilized to determine when asymmetric step lengths may be causing chronic overloading of a particular leg of the individual when compared to the other leg of the individual. Such determinations may then allow for taking preventative and/or corrective measures associated with chronic overloading and any possible resulting issues (e.g., osteoarthritis).

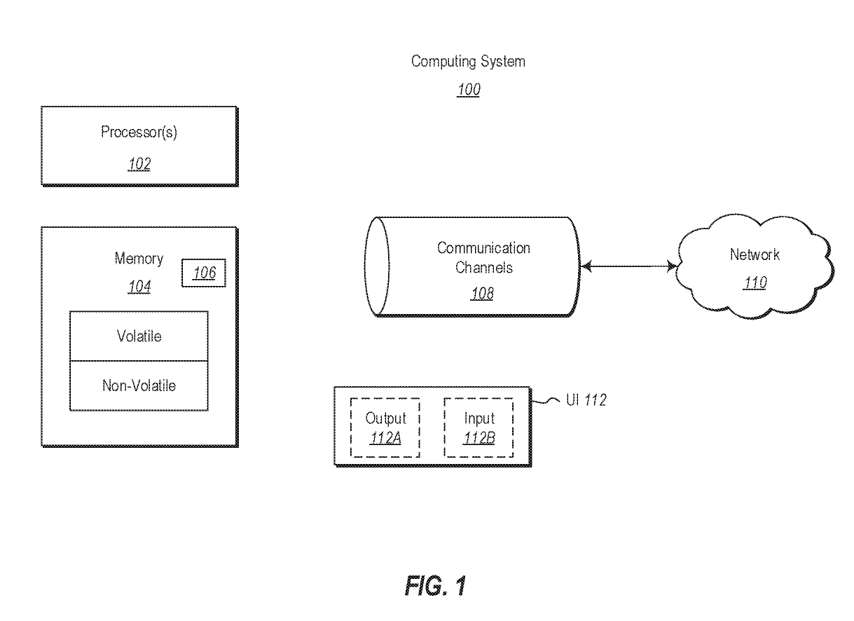

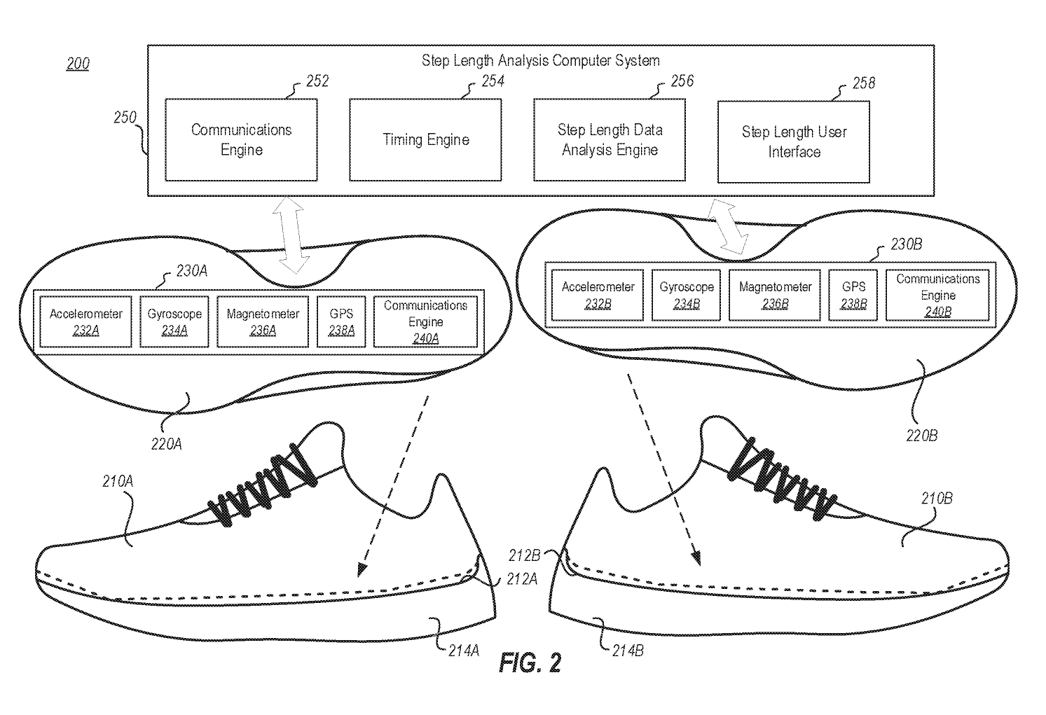

Some introductory discussion of a computing system will be described with respect to FIG. 1. Then determining step lengths of an individual will be described with respect to FIGS. 2 through 6.

Computing systems are now increasingly taking a wide variety of forms. Computing systems may, for example, be handheld devices, appliances, laptop computers, desktop computers, mainframes, distributed computing systems, datacenters, or even devices that have not conventionally been considered a computing system, such as wearables (e.g., glasses). In this description and in the claims, the term “computing system” is defined broadly as including any device or system (or combination thereof) that includes at least one physical and tangible processor, and a physical and tangible memory capable of having thereon computer-executable instructions that may be executed by a processor. The memory may take any form and may depend on the nature and form of the computing system. A computing system may be distributed over a network environment and may include multiple constituent computing systems.

As illustrated in FIG. 1, in its most basic configuration, a computing system 100 typically includes at least one hardware processing unit 102 and memory 104. The memory 104 may be physical system memory, which may be volatile, non-volatile, or some combination of the two. The term “memory” may also be used herein to refer to non-volatile mass storage such as physical storage media. If the computing system is distributed, the processing, memory and/or storage capability may be distributed as well.

The computing system 100 also has thereon multiple structures often referred to as an “executable component”. For instance, the memory 104 of the computing system 100 is illustrated as including executable component 106. The term “executable component” is the name for a structure that is well understood to one of ordinary skill in the art in the field of computing as being a structure that can be software, hardware, or a combination thereof. For instance, when implemented in software, one of ordinary skill in the art would understand that the structure of an executable component may include software objects, routines, methods, and so forth, that may be executed on the computing system, whether such an executable component exists in the heap of a computing system, or whether the executable component exists on computer-readable storage media.

In such a case, one of ordinary skill in the art will recognize that the structure of the executable component exists on a computer-readable medium such that, when interpreted by one or more processors of a computing system (e.g., by a processor thread), the computing system is caused to perform a function. Such structure may be computer-readable directly by the processors (as is the case if the executable component were binary). Alternatively, the structure may be structured to be interpretable and/or compiled (whether in a single stage or in multiple stages) so as to generate such binary that is directly interpretable by the processors. Such an understanding of example structures of an executable component is well within the understanding of one of ordinary skill in the art of computing when using the term “executable component”.

The term “executable component” is also well understood by one of ordinary skill as including structures that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. In this description, the terms “component”, “service”, “engine”, “module”, “control”, or the like may also be used. As used in this description and in the case, these terms (whether expressed with or without a modifying clause) are also intended to be synonymous with the term “executable component”, and thus also have a structure that is well understood by those of ordinary skill in the art of computing.

In the description that follows, embodiments are described with reference to acts that are performed by one or more computing systems. If such acts are implemented in software, one or more processors (of the associated computing system that performs the act) direct the operation of the computing system in response to having executed computer-executable instructions that constitute an executable component. For example, such computer-executable instructions may be embodied on one or more computer-readable media that form a computer program product. An example of such an operation involves the manipulation of data.

The computer-executable instructions (and the manipulated data) may be stored in the memory 104 of the computing system 100. Computing system 100 may also contain communication channels 108 that allow the computing system 100 to communicate with other computing systems over, for example, network 110.

While not all computing systems require a user interface, in some embodiments, the computing system 100 includes a user interface 112 for use in interfacing with a user. The user interface 112 may include output mechanisms 112A as well as input mechanisms 112B. The principles described herein are not limited to the precise output mechanisms 112A or input mechanisms 112B as such will depend on the nature of the device. However, output mechanisms 112A might include, for instance, speakers, displays, tactile output, holograms and so forth. Examples of input mechanisms 112B might include, for instance, microphones, touchscreens, holograms, cameras, keyboards, mouse of other pointer input, sensors of any type, and so forth.

Embodiments described herein may comprise or utilize a special purpose or general-purpose computing system including computer hardware, such as, for example, one or more processors and system memory, as discussed in greater detail below. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: storage media and transmission media.

Computer-readable storage media includes RAM, ROM, EEPROM, CD-ROM or other optical disk storage, magnetic disk storage or other magnetic storage devices, or any other physical and tangible storage medium which can be used to store desired program code means in the form of computer-executable instructions or data structures and which can be accessed by a general purpose or special purpose computing system.

A “network” is defined as one or more data links that enable the transport of electronic data between computing systems and/or modules and/or other electronic devices. When information is transferred or provided over a network or another communications connection (either hardwired, wireless, or a combination of hardwired or wireless) to a computing system, the computing system properly views the connection as a transmission medium. Transmissions media can include a network and/or data links which can be used to carry desired program code means in the form of computer-executable instructions or data structures and which can be accessed by a general purpose or special purpose computing system. Combinations of the above should also be included within the scope of computer-readable media.

Further, upon reaching various computing system components, program code means in the form of computer-executable instructions or data structures can be transferred automatically from transmission media to storage media (or vice versa). For example, computer-executable instructions or data structures received over a network or data link can be buffered in RAM within a network interface module (e.g., a “NIC”), and then eventually transferred to computing system RAM and/or to less volatile storage media at a computing system. Thus, it should be understood that storage media can be included in computing system components that also (or even primarily) utilize transmission media.

Computer-executable instructions comprise, for example, instructions and data which, when executed at a processor, cause a general purpose computing system, special purpose computing system, or special purpose processing device to perform a certain function or group of functions. Alternatively, or in addition, the computer-executable instructions may configure the computing system to perform a certain function or group of functions. The computer executable instructions may be, for example, binaries or even instructions that undergo some translation (such as compilation) before direct execution by the processors, such as intermediate format instructions such as assembly language, or even source code.

Those skilled in the art will appreciate that the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, datacenters, wearables (such as glasses) and the like. The invention may also be practiced in distributed system environments where local and remote computing systems, which are linked (either by hardwired data links, wireless data links, or by a combination of hardwired and wireless data links) through a network, both perform tasks. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

Computer systems and related technology, as described herein, now affect many aspects of society. Indeed, the computer system's ability to process information has transformed the way we live and work. Computer systems now commonly perform a host of tasks (e.g., word processing, scheduling, accounting, etc.) that prior to the advent of the computer system were performed manually. More recently, computer systems have been coupled to one another and to other electronic devices to form both wired and wireless computer networks over which the computer systems and other electronic devices can transfer electronic data.

In addition, computing systems (including sensors) are often being coupled to humans to allow for performing a variety of tasks. For instance, computer systems are now being used for identification of individuals (e.g., biometrics, including finger print scanners, retina scanners, facial recognition, and so forth), for health monitoring (e.g., heart rate sensors, pulse oximeters, and so forth), and a variety of other users. Such technology can be instrumental in identifying and/or preventing serious medical issues from occurring or worsening.

Notably, asymmetric step lengths (and/or asymmetric gait) can be common in individuals with unilateral lower limb amputations, post cerebral accident victims, stroke victims, lower limb orthopedic surgery patients, and so forth. Asymmetry of step lengths refers to a difference in distance from a left heel strike to a right heel strike when walking/running in comparison to a distance from a right heel strike to a left heel strike when walking/running, as further described herein. Asymmetric step length has been linked with chronic overloading in the non-amputated limb. Such overloading can occur because less time is often spent on the amputated leg, as a shorter step length is often taken on the intact leg.

Additionally, step length asymmetry has been shown to increase with walking speed which can account for a greater loading of joints in an intact limb of a unilateral amputee. Such asymmetry of step lengths, and subsequent overloading of the non-amputated limb, has been associated with osteoarthritis in the knee of the non-amputated limb, which can have a profound impact on an individual's quality of life. Research suggests this higher prevalence of osteoarthritis may be due to an increased force in the intact leg of a unilateral amputee, which results from asymmetrical step lengths (and/or asymmetrical gait).

Accordingly, FIG. 2 illustrates an environment 200 for determining step lengths of an individual, which can then be used to identify asymmetric step lengths and take corrective/preventative measure to mitigate issues (e.g., osteoarthritis) resulting from asymmetric step lengths. As illustrated, the environment 200 includes a left shoe 210A, a right shoe 210B, a left insole 220A, a right insole 220B, and a step length analysis computer system 250. The shoes 210 (i.e., the left shoe 210A and the right shoe 210B) may comprise any type of applicable footwear that can be worn on a natural foot or a prosthetic foot of an individual. For instance, the shoes 210 may comprise athletic shoes, dress shoes, boots, and so forth. As shown, the shoes 210 include an insole receiving portion 212 (i.e., insole receiving portion 212A and insole receiving portion 212B) for receiving the insoles 220 (i.e., the left insole 220A and the right insole 220B), as well as soles 214 (i.e., sole 214A and sole 214B).

Accordingly, the insoles 220 can be sized and shaped to fit within the shoes 210. In an example, the insoles 220 may comprise an elastomeric and/or polymeric material such as rubberized silicone. Additionally, as illustrated, the soles 220 include a number of components 230 that are used in determining a step length. More specifically, the components 230 may include accelerometers 232 (i.e., accelerometer 232A and accelerometer 232B), gyroscopes 234 (i.e., gyroscope 234A and gyroscope 234B), magnetometers 236 (i.e., magnetometer 236A and magnetometer 236B), global positioning system (GPS) devices 238 (i.e., the GPS 238A and the GPS 238B), and communications engine 240 (i.e., communications engine 240A and communications engine 240B). In particular, the components 230 may, along with the step length analysis computer system 250, be configured to determine step lengths of an individual, as further described herein.

Additionally, each of the components 230 may comprise essentially any type of component known by one of skill in the art. For instance, any particular type of accelerometers 232 (e.g., one-axis accelerometers, two-axis accelerometers, three-axis accelerometers, and so forth), gyroscopes 234 (e.g., one-axis gyroscopes, two-axis gyroscopes, three-axis gyroscopes, and so forth), magnetometers 236 (e.g., one-axis magnetometers, two-axis magnetometers, three-axis magnetometers, and so forth), any type of appropriate GPS device, and so forth may be used to practice the principles described herein. In a specific example, two accelerometer axes and one gyroscope axis may be used to identify a heel strike and/or a toe-off (i.e., when the toes of a foot have been lifted off the ground after making contact with the ground) when walking on flat or inclined surfaces. With respect to identifying heel strikes and/or toe-offs when walking up or down stairs, additional gyroscope and accelerometer axes may be utilized.

Notably, while the components 230 are shown as being positioned within the insoles 220 (and ultimately the shoes 210), the principles described herein may be practiced by utilizing many different components. For instance, some configurations may include more, and/or different, components than those illustrated as the being included within the components 230 (e.g., force sensors, bilateral force sensors, strain gages, pedometers, levels, Reed switches, Hall Effect sensors, tilt/inclination sensors, and so forth), while other configurations may include less, and/or different, components than those illustrated and discussed herein. In some embodiments, the components 230 can be configured to be positioned within commercially available shoes already obtained by a user, such as with a sensor insert or the insole 220. Alternatively, the components 230 can be provided in a custom shoe or foot-borne device specifically made for the components/sensors and/or the particular user. Additionally, or alternatively, the insoles 220 can replace original insoles of the shoes 210.

Furthermore, while the various components 230 are illustrated in FIG. 2 as being included within or on the insoles 220, the components 230 may be included in any location with respect to the shoes 210 or a foot of an individual. For instance, in some embodiments, one or more of the components 230 (e.g., 222, 225, and so forth) may be located within a sole 214 (e.g., sole 214A and sole 214B) of the shoes 210, located within in a lining (not shown) of the shoes 210, coupled to laces (not shown) of the shoes 210, coupled to an outer portion of the shoes 210, coupled to and/or located within a sock of an individual, and so forth. Additionally, while all the components 230 are shown as being located in a single location (i.e., the insoles 220), in some embodiments, one or more first components may be positioned at a first location (e.g., the sole 214) of the shoes 220, while one or more second components are positioned at one or more second locations (e.g., insole 220, laces, socks, and so forth).

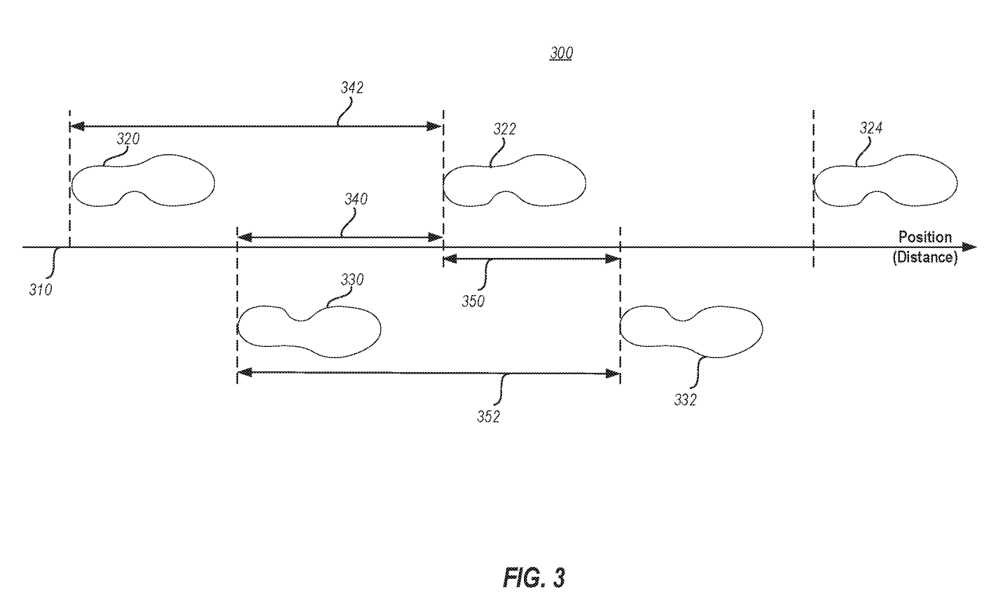

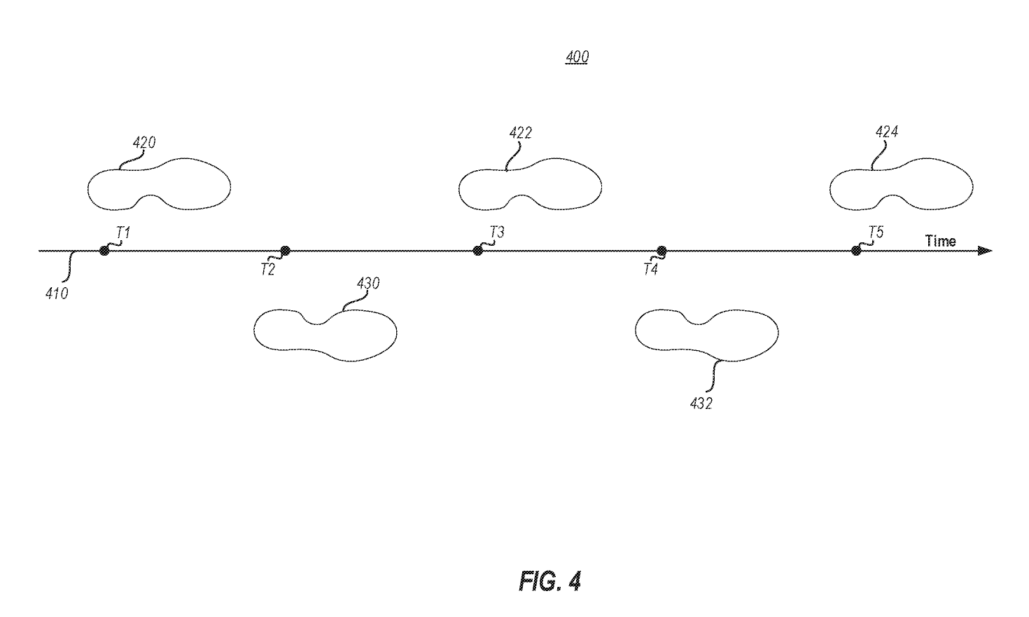

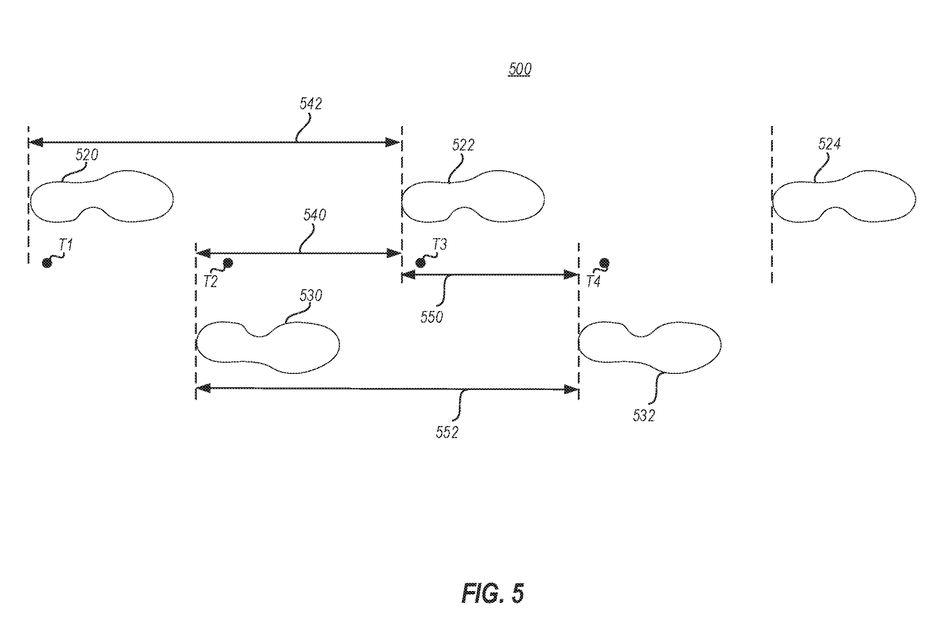

As briefly discussed, the components 230 may aid the step length analysis computer system 250 in determining step lengths of an individual. In particular, the accelerometers 232, the gyroscopes 234, and the magnetometers 236 may be configured to work in combination to determine when a heel strike of an individual has occurred (i.e., when a heel of an individual has made contact with the ground), as further described with respect to FIG. 3. As shown, FIG. 3 illustrates a series of detected left heel strikes based on a series of left stances (i.e., left stance 320, left stance 322, and left stance 324) and a series of right heel strikes based on a series of right stances (i.e., right stance 330 and right stance 332). These detected heel strikes (e.g., left heel strike 320, right heel strike 330, and so forth) may then be used to determine step lengths associated with an individual, as further described with respect to FIGS. 4 and 5.

Notably, each of the stances (e.g., the left stance 320, the right stance 330, and so forth) are shown as being along an x-axis 310 associated with distance. More specifically, the x-axis (i.e., distance) is included in FIG. 3 to illustrate that both stride lengths (i.e., left stride length 342 and right stride length 352) for each foot and step lengths (i.e., left step length 340 and right step length 350) for each foot may be determined utilizing the principles described herein. However, the disclosure presented herein is particularly focused on determining step lengths, as further described herein. In particular, stride lengths are referred to herein as the length from a first left heel strike to a second left heel strike (i.e., left stride length 342) when walking/running or a first right heel strike to a second right heel strike (i.e., right stride length 352) when walking/running. Step lengths, on the other hand, comprise a distance from a right heel strike to a left heel strike (i.e., left step length 340) when walking/running or a left heel strike to a right heel strike (i.e., right step length 350) when walking/running.

As illustrated, the components 230 of FIG. 2 also include the GPS devices 238 and the communications engines 240. The GPS devices 238 may be configured to determine the foot speed of each foot of an individual, as further described with respect to FIG. 5, while the communications engines 240 may comprise any combination of hardware and/or software that are configured to communicate with the communications engine 252 of the step length analysis computer system 250. In an example, the communications engines 240 may include a wireless transmitter and receiver that are configured to wirelessly communicate with the communications engine 252.

As briefly discussed, the environment 200 also includes the step length analysis computer system 250. The step length analysis computer system may correspond to the computer system 100, as described with respect to FIG. 1. The step length analysis computer system 250 may comprise any type of computer system that is configured to aid in determining a step length of an individual, as further described herein. In an example, the step length analysis computer system 250 may comprise a desktop computer, a laptop computer, a tablet, a smartphone, and so forth. Furthermore, the free hand conversion computer system 210 may be running any applicable operating system, including but not limited to, MICROSOFT® WINDOWS®, APPLE® MACOS®, APPLE IOS®, GOOGLE™ CHROME OS™, ANDROID™, and so forth.

As shown, the step length analysis computer system 250 may include various engines, functional blocks, and components, including communications engine 252, timing engine 254, step length data analytics engine 256, and the step length user interface 258. The various engines, components, and/or functional blocks of the step length analysis computer system 250 may be implemented on a local computer system or may be implemented on a distributed computer system that includes elements resident in the cloud or that implement aspects of cloud computing (i.e., at least one of the various illustrated engines may be implemented locally, while at least one other engine may be implemented remotely).

Alternatively, or additionally, one or more of the engines of the step length analysis computer system may be located remote from the components 230 and/or shoes 210, such as on a hip or belt worn housing. Alternatively, or additionally, one or more of the engines of the step length analysis computer system may be located in or on the shoe, or around an ankle or prosthesis of an individual. Alternatively, or additionally, one or more of the engines of the step length analysis computer system may be formed together on a circuit within the shoe or insole. Alternatively, or additionally, one or more of the engines of the step length analysis computer system may be located in a wrist-worn device or in a housing that can be put in a pocket of a clothing item of an individual.

While the illustrated engines (i.e., the communications engine 252, the timing engine 254, the step length data analytics engine 256, and the step length user interface 258) of the step length analysis computer system 250 are shown as being part of a separate computer system (i.e., the step length analysis computer system 250), one or more of the illustrated engines may be included as part of the footwear (e.g., within the shoes 210, within or coupled to the insoles 220, coupled to socks, coupled to laces, and so forth) of an individual, as further described herein with respect to the components 230. The various engines, functional blocks, and/or components of the free hand conversion computer system 210 may be implemented as software, hardware, or a combination of software and hardware.

Notably, the configuration of step length analysis computer system 250 (and the components 230) illustrated in FIG. 2 is shown only for exemplary purposes. As such, the step length analysis computer system 250 (and the components 230) may include more or less than the engines, functional blocks, and/or components illustrated in FIG. 2. Although not illustrated, the various engines of the step length analysis engine computer system 250 (and the components 230) may access and/or utilize a processor and memory, such as the processor 102 and the memory 104 of FIG. 1, as needed to perform their various functions.

As briefly described, the step length analysis computer system includes the communications engine 252, the timing engine 254, the step length data analytics engine 256, and the step length user interface 258. The communications engine 252 may comprise any combination of hardware and/or software that is configured to communicate with the communications engine 240 of the components 230. The timing engine 254 may be responsible for determining a relative start time that is used for determining heel strikes for both a left foot and right foot of an individual (and ultimately, for determining a left step length and a right step length for an individual/user), as further described with respect to FIGS. 4 and 5.

In some embodiments, the timing engine may include a real-time clock at the footwear of an individual (e.g., the shoes 210, the insoles 230, laces, socks, and so forth), which real-time clocks may then be synced with respect to one another (i.e., a left foot clock with a right foot clock) for determining a relative time at which each identified heel strike has occurred, as discussed further herein. In other embodiments, the timing engine may include any appropriate hardware and/or software at the footwear of an individual (e.g., the shoes 210, the insoles 230, laces, socks, and so forth) for communicating with each other such that a relative time may be determined (e.g., an agreement that a certain time is to be considered time zero as a reference point for determining a point in time associated with each identified heel strike).

The step length data analytics engine 256 may be configured to analyze the data (e.g., heel strike data) gathered by the components 230, analyze determined relative timing data, associate relative timing to each detected heel strike, determine step lengths associated with an individual, determine stride lengths associated with an individual, determine asymmetry of step lengths associated with an individual, and so forth, as further described with respect to FIG. 5. In an example, the step length data analytics engine may comprise a computer, at least a portion of a hardware and/or software processor of a computer, a microprocessor, a microcontroller, and so forth.

Finally, the step length user interface 258 may comprise any appropriate user interface (e.g., a user interface associated with an application) that allows for presenting information associated with data gathered and analyzed by the components 230 and the various engines of the step length analysis computer system 250. For instance, an application including the step length user interface 258 may allow an individual to view foot speed, step lengths, stride lengths, and so forth associated with a particular user of footwear as shown and described with respect to the environment 200 of FIG. 2.

As briefly discussed, FIG. 4 illustrates a number of detected heel strikes (e.g., left heel strike 420, right heel strike 430, and so forth) associated with footsteps of an individual along an x-axis 410 associated with time. Each detected heel strike, regardless of whether the detected heel strike is a left heel strike or a right heel strike, may then be associated with a particular point in time associated with the relative start time determined by the timing engine. For instance, the detected left heel strike 420 may be associated with a time T1 that corresponds to the relative time determined by the timing engine. Similarly, the right heel strike 430, the left heel strike 422, the right heel strike 432, and the left heel strike 424 may be associated with a time T2, a time T3, a time T4, and a time T5, respectively, that are each associated with the relative time determined by the timing engine (i.e., demonstrated by each detected heel strike being associated with the same timeline 410).

In a more specific example, time T1 may be at 0 seconds, time T2 may be at 0.5 seconds, time T3 may be at 1.2 seconds, and so forth, along the same timeline 410. In another specific example, time T1 may be at 0.3 seconds, time T2 may be at 0.9 seconds, time T3 may be at 1.7 seconds, and so forth, along the same timeline 410. Accordingly, the relative timing determined by the timing engine 254 may be communicated to any of the components 230 and/or the step length data analytics engine for proper determinations of when heel strikes have occurred with respect to the determined relative timing. In some embodiments, the determined relative time and each detected heel strike may be communicated to the step length data analytics engine. The step length data analytics engine may then associate each particular detected heel strike with a point in time corresponding to the determine relative time. In other embodiments, detected heel strike data, including an associated timing of each heel strike that corresponds to the relative timing determined by the timing engine, may be provided to the step length data analytics engine for further analysis associated with determining step lengths.

Accordingly, FIG. 5 illustrates detected heel strikes associated with both points in time that the heel strikes occurred, as well as distances associated with step lengths. As shown, FIG. 5 again includes a series of left heel strikes (i.e., left heel strike 520, left heel strike 522, and left heel strike 524) associated with a series of left footsteps, a series of right heel strikes (i.e., right heel strike 530 and right heel strike 532) associated with a series of right footsteps, a left step length 540, a left stride length 542, a right step length 550, a right stride length 552, and a series of points in time (i.e., T1 through T4) that are each associated with a point in time in which a heel strike is detected.

With respect to determining both the left step length 540 and the right step length 550, the timing engine 254 may initially determine a relative time that may be utilized to associate each detected heel strike with a point in time that refers to the determined relative time. For instance, the step length data analytics engine may utilize the determined relative time and heel strike data generated by the accelerometers 222, the gyroscopes 224, and the magnetometers 226 (or any other appropriate combination of sensors known in the art for detecting a heel strike) associated with each detected heel strike (i.e., left heel strike 520, left heel strike 522, left heel strike 524, right heel strike 530, and right heel strike 532) to determine that the left heel strike 520 is associated with a point in time T1, the right heel strike 530 is associated with a point in time T2, the left heel strike 522 is associated with a point in time T3, the right heel strike 532 is associated with a point in time T4, and so forth.

Additionally, as briefly described herein, the GPS devices 238 may be used to determine a left foot speed associated with both a left foot stride length 542 (i.e., from the left heel strike 530 to the left heel strike 532) and a timing of the left foot stride (i.e., from T1 to T3), as well as a right foot speed associated with both a right foot stride length 552 (i.e., from the right heel strike 540 to the right heel strike 542) and a timing of the right foot stride (i.e., from T2 to T4). More specifically, the GPS device may determine a left foot speed by determining a length of the left foot stride 542 and dividing the length of the left foot stride by the result of T1 subtracted from T3, and a right foot speed by determining a length of the right foot stride 552 and dividing the length of the right foot stride by the result of T2 subtracted from T4.

In a particular example, assume that the left stride length 542 is 1 meter, the right stride length 552 is 1.2 meter, the point in time T1 is at 1 second, the point in time T2 is at 1.4 seconds, the point in time T3 is at 1.9 seconds, and the point in time T4 is 2.3 seconds. In such an example, the GPS devices 238 may determine that the left foot speed is approximately 1.1 meters per second (i.e., 1 meter divided by the result of 1.9 seconds minus 1 second, or 0.9 seconds) and the right foot speed is approximately 1.3 meters per second (i.e., 1.2 meter divided by the result of 2.3 seconds minus 1.4 seconds, or 0.9 seconds). Notably, while a GPS device is discussed as being utilized to determine both a left and a right foot speed, any device that is capable of determining a foot speed that is known in the art may be used to practice the principles described herein.

Upon receiving data associated with the left foot speed, the right foot speed, the heel strike data, and the determined relative timing for detected heel strike data, the step length data analytics engine 256 may determine the left step length 540 and the right step length 550. Using the previous example, the step length data analytics engine may utilize the determined left foot speed of 1.1 meters per second and the timing between the right heel strike 530 and the left heel strike 522 (i.e., T3 minus T2) to determine the left step length 540. More specifically, the step length data analytics engine 256 may multiply the left foot speed of 1.1 meters per second by 0.5 seconds (i.e., T3 minus T2, or 1.9 seconds minus 1.4 seconds) to determine that the left step length 540 is approximately 0.55 meters. Similarly, the step length data analytics engine may utilize the determined right foot speed of 1.3 meters per second and the timing between the left heel strike 522 and the right heel strike 532 (i.e., T4 minus T3) to determine the right step length 550. More specifically, the step length data analytics engine 256 may multiply the right foot speed of 1.3 meters per second by 0.4 seconds (i.e., T4 minus T3, or 2.3 seconds minus 1.9 seconds) to determine that the right step length 550 is approximately 0.52 meters.

In addition to step length, step length symmetry (i.e., whether step lengths are approximately the same or within a threshold value of being the same) may also be determined (e.g., via the step length data analytics engine 256). In an example, step length symmetry may be calculated as a ratio (i.e., a left step length of an individual divided by a right step length of the individual, and vice versa). In another example, step length symmetry may be calculated as a relative percentage of step lengths of the individual (i.e., a left step length divided by a sum of the left step length plus the right step length of the individual or a right step length divided by the sum of the left step length plus the right step length of the individual).

Notably, such determinations (e.g., step length determinations, step length symmetry determinations, and so forth) may be performed (e.g., by the step length data analytics engine 256) in real-time (i.e., immediately, or almost immediately, upon gathering step length data—relative time, identified heel strikes, and so forth). In other embodiments, such determinations may be performed at a later time.

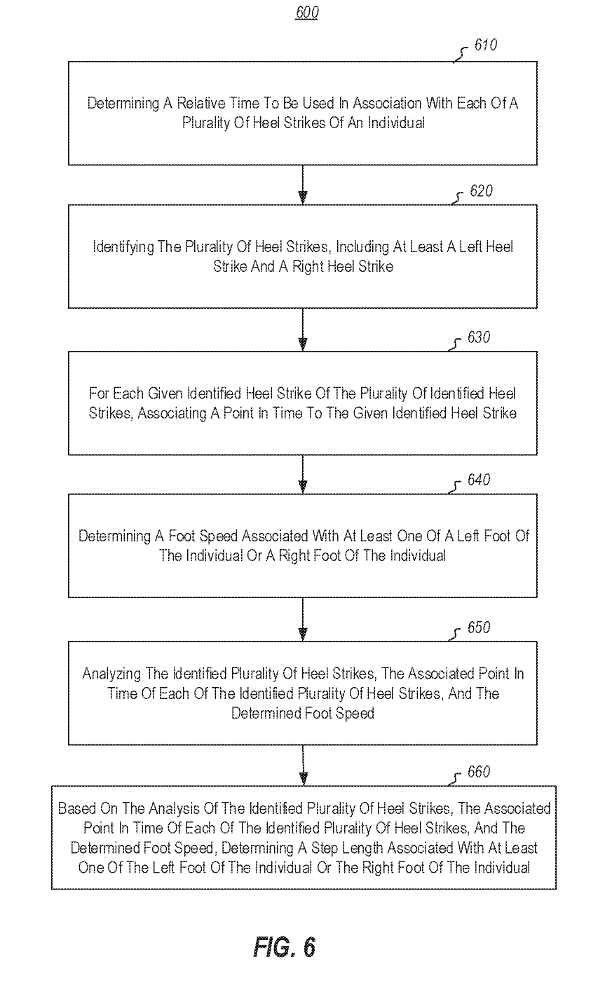

FIG. 6 illustrates a flowchart of a method 600 for determining a step length of an individual. The method 600 is described with frequent reference to the environments of FIGS. 2-5. The method 600 includes, determining a relative time to be used in association with each of a plurality of heel strikes of an individual (Act 610). For example, a relative time may be determined such that a series of identified heel strikes are determined to have occurred at points in time T1, T2, T3, T4, and T5, as shown in FIG. 4 (and generally shown in FIG. 5). The method 600 further includes identifying the plurality of heel strikes, including at least a left heel strike and a right heel strike (Act 620). For example, FIGS. 3 through 5 each show a plurality of identified heel strikes (e.g., the heel strike 320, the heel strike 330, and so forth) associated with an individual that is walking/running while utilizing the environment 200 illustrated in FIG. 2.

The method 600 further includes, for each given identified heel strike of the plurality of identified heel strikes, associating a point in time to the given identified heel strike (Act 630). The point in time corresponds to the determined relative time. For instance, FIG. 5 shows that the identified heel strike 520 is associated with the point in time T1, the identified heel strike 530 is associated with the point in time T2, and so forth, wherein T1, T2, and so forth are each point in time that correspond to the determined relative time. The method 600 also includes determining a foot speed associated with at least one of a left foot of the individual or a right foot of the individual (Act 640). For instance, the GPS devices 238 may determine a foot speed associated with walking/running of an individual. As described more fully herein, the foot speed may correspond to a stride associated with the left foot or a stride associated with the right foot.

The method 600 further includes, analyzing the identified plurality of heel strikes, the associated point in time of each of the identified plurality of heel strikes, and the determined foot speed (Act 650). For instance, as further described with respect to FIG. 5, a determined relative time, identified heel strike data, and determined foot speed data may be provided to the step length data analytics engine 256. The method 600 also includes, based on the analysis of the identified plurality of heel strikes, the associated point in time of each of the identified plurality of heel strikes, and the determined foot speed, determining a step length associated with at least one of the left foot of the individual or the right foot of the individual (Act 660). For example, as further described with respect to FIGS. 2 and 5, the step length data analytics engine 256 may utilize a determined relative time, identified heel strike data, and determined foot speed data to determine a step length associated with a left foot and/or right foot of an individual that is utilizing the environment 200.

In this way, one or more components for determining step length may be coupled to an individual via footwear (e.g., shoes, boots, socks, and so forth) of the individual. The one or more components and potentially, one or more components separate from the footwear, may gather and analyze data associated with a determined foot speed of the individual, identified heel strikes of the individual, and a determined relative time for determining when the identified heel strikes occurred in relation to other identified heel strikes. This data may then be analyzed to determine both a left step length of the individual, as well as a right step length of the individual. The determined step lengths may then be utilized to determine when asymmetric step lengths may be causing chronic overloading of a particular leg of the individual when compared to the other leg of the individual. Such determinations may then allow for taking preventative and/or corrective measures associated with chronic overloading and any possible resulting issues (e.g., osteoarthritis).

Although the subject matter has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. A computer system comprising:
one or more processors; and
one or more computer-readable storage media having stored thereon computer-executable instructions that are executable by the one or more processors to cause the computer system to determine a step length of an individual, the computer-executable instructions including instructions that are executable to cause the computer system to perform at least the following:
determine a relative time to be used in association with each of a plurality of heel strikes of an individual;
identify the plurality of heel strikes, including at least a left heel strike and a right heel strike;
for each given identified heel strike of the plurality of identified heel strikes, associate a point in time to the given identified heel strike, the point in time corresponding to the determined relative time;
determine a foot speed associated with at least one of a left foot of the individual or a right foot of the individual, wherein the foot speed corresponds to a stride associated with the left foot or a stride associated with the right foot;
analyze the identified plurality of heel strikes, the associated point in time of each of the identified plurality of heel strikes, and the determined foot speed; and
based on the analysis of the identified plurality of heel strikes, the associated point in time of each of the identified plurality of heel strikes, and the determined foot speed, determine a step length associated with at least one of the left foot of the individual or the right foot of the individual.

2. The computer system in accordance with claim 1, wherein the plurality of identified heel strikes includes at least two left heel strikes and at least two right heel strikes.

3. The computer system in accordance with claim 1, wherein the stride associated with the left foot comprises a distance from a first heel strike of the left foot to a second heel strike of the left foot and the stride associated with the right foot comprises a distance from a first heel strike of the right foot to a second heel strike of the right foot.

4. The computer system in accordance with claim 1, wherein the step length associated with the left foot comprises a distance from a right heel strike to a next left heel strike and the step length associated with the right foot comprises a distance from a left heel strike to a next right heel strike.

5. The computer system in accordance with claim 1, wherein the foot speed is determined using a Global Positioning System (GPS) device.

6. The computer system in accordance with claim 1, wherein each heel strike is identified using at least one axis of an accelerometer, a gyroscope, or a magnetometer.

7. The computer system in accordance with claim 1, wherein at least one component used to identify the plurality of heel strikes is located within a sole of footwear of the individual.

8. A method, implemented at a computer system that includes one or more processors, for determining a step length of an individual, comprising:
determining a relative time to be used in association with each of a plurality of heel strikes of an individual;
identifying the plurality of heel strikes, including at least a left heel strike and a right heel strike;
for each given identified heel strike of the plurality of identified heel strikes, associating a point in time to the given identified heel strike, the point in time corresponding to the determined relative time;
determining a foot speed associated with at least one of a left foot of the individual or a right foot of the individual, wherein the foot speed corresponds to a stride associated with the left foot or a stride associated with the right foot;
analyzing the identified plurality of heel strikes, the associated point in time of each of the identified plurality of heel strikes, and the determined foot speed; and
based on the analysis of the identified plurality of heel strikes, the associated point in time of each of the identified plurality of heel strikes, and the determined foot speed, determining a step length associated with at least one of the left foot of the individual or the right foot of the individual.

9. The method in accordance with claim 8, wherein the plurality of identified heel strikes includes at least two left heel strikes and at least two right heel strikes.

10. The method in accordance with claim 8, wherein the stride associated with the left foot comprises a distance from a first heel strike of the left foot to a second heel strike of the left foot and the stride associated with the right foot comprises a distance from a first heel strike of the right foot to a second heel strike of the right foot.

11. The method in accordance with claim 8, wherein the step length associated with the left foot comprises a distance from a right heel strike to a next left heel strike and the step length associated with the right foot comprises a distance from a left heel strike to a next right heel strike.

12. The method in accordance with claim 8, wherein the foot speed is determined using a Global Positioning System (GPS) device.

13. The method in accordance with claim 8, wherein each heel strike is identified using at least one axis of an accelerometer, a gyroscope, or a magnetometer.

14. The method in accordance with claim 8, wherein at least one component used to identify the plurality of heel strikes is located within a sole of footwear of the individual.

15. A computer program product comprising one or more computer readable media having stored thereon computer-executable instructions that are executable by one or more processors of a computer system to cause the computer system to determine a step length of an individual, the computer-executable instructions including instructions that are executable to cause the computer system to perform at least the following:
determine a relative time to be used in association with each of a plurality of heel strikes of an individual;
identify the plurality of heel strikes, including at least a left heel strike and a right heel strike;
for each given identified heel strike of the plurality of identified heel strikes, associate a point in time to the given identified heel strike, the point in time corresponding to the determined relative time;
determine a foot speed associated with at least one of a left foot of the individual or a right foot of the individual, wherein the foot speed corresponds to a stride associated with the left foot or a stride associated with the right foot;
analyze the identified plurality of heel strikes, the associated point in time of each of the identified plurality of heel strikes, and the determined foot speed; and
based on the analysis of the identified plurality of heel strikes, the associated point in time of each of the identified plurality of heel strikes, and the determined foot speed, determine a step length associated with at least one of the left foot of the individual or the right foot of the individual.

16. The computer program product in accordance with claim 15, wherein the plurality of identified heel strikes includes at least two left heel strikes and at least two right heel strikes.

17. The computer program product in accordance with claim 15, wherein the stride associated with the left foot comprises a distance from a first heel strike of the left foot to a second heel strike of the left foot and the stride associated with the right foot comprises a distance from a first heel strike of the right foot to a second heel strike of the right foot.

18. The computer program product in accordance with claim 15, wherein the step length associated with the left foot comprises a distance from a right heel strike to a next left heel strike and the step length associated with the right foot comprises a distance from a left heel strike to a next right heel strike.

19. The computer program product in accordance with claim 15, wherein the foot speed is determined using a Global Positioning System (GPS) device.

20. The computer program product in accordance with claim 15, wherein each heel strike is identified using at least one axis of an accelerometer, a gyroscope, or a magnetometer.
